(19) United States
(12) Patent Application Publication
(10) Pub. No.: US 2024/0016743 A1
(43) Pub. Date: Jan. 18, 2024

(54) A METHOD FOR PRODUCING ENTACAPONE TABLETS

(71) Applicant: CHINA MEHECO KANGLI PHARMA CO., LTD, Haikou City (CN)

(72) Inventors: Yonghong Fu, Haikou Ciry (CN); Haixiong Zhong, Haikou City (CN); Fangyu Lin, Haikou City (CN); Hanwen Xiao, Haikou City (CN); Yuannu Zheng, Haikou City (CN); Xiaoxue Lin, Haikou City (CN); Xiongzhao Tang, Haikou City (CN)

(21) Appl. No.: 17/297,283

(22) PCT Filed: May 13, 2021
(86) PCT No.: PCT/CN2021/093467

§ 371 Date: May 26, 2021

(30) Foreign Application Priority Data
Mar. 31, 2021 (CN) 202110343909.7

(51) Int. Cl. A61K 9/20

(52) U.S. Cl. CPC A61K 9/2018; A61K 9/2054

(57) ABSTRACT

A method for producing entacapone tablets, including auxiliary materials passing screening, batching, mixing, sieving, mixing, drying granulation, sieving, mixing with magnesium stearate and pressing finished products. Mannitol, a supplementary material, was prepared by following steps: the mixture of fructose and glucose was hydrolyzed from sucrose; The glucose obtained was further hydrolyzed to obtain a mixture of mannose, fructose and glucose; The mannitol product is obtained by mixing the mannose, fructose and glucose obtained in steps 1 and 2, hydrogenation, refining, concentration, crystallization and pressure filtration; The finished mannitol product is dried and crushed by mixing with water mixed with binder. The compressed mannitol particles with a particle size of 200-350 um are obtained, which are used as auxiliary materials for the preparation of entacapone tablets. The entacapone tablet prepared by the production method of the entacapone tablet provided by the invention has stable structure, stable physical properties and long shelf life

### FIELD OF THE INVENTION

The invention relates to the technical field of the production of neurodrugs, in particular to a production method of entacapone tablets.

### BACKGROUND OF THE INVENTION

The chemical name of entacapone is (E)-2-cyano-3-(3,4-dihydroxy-5-nitrophenyl)-N, N-diethyl-2-acrylamide, molecular formula: C₁₄H₁₅N₃O₅, molecular weight: 305.28. It belongs to catechol-O-methyltransferase (COMT) inhibitor. It is a reversible, specific and mainly acts on peripheral COMT inhibitor. It is used together with levodopa preparation.

Clinical trials showed that levodopa could prolong the “On” time by 16% and shorten the “Off” time by 24%. This product mainly inhibits COMT in surrounding tissues. The inhibition of COMT in red blood cells is closely related to the plasma concentration of this product, which reflects the reversibility of COMT inhibition.

There are many forms of entacapone. In the existing production methods of tablet drugs, most raw materials and excipients are purchased from third-party enterprises, and there is no complete production process of entacapone tablets, which leads to that the tablets are not easy to be pressed, the finished products are easy to be broken, and the physical properties are unstable.

### SUMMARY OF THE INVENTION

In order to solve the shortcomings of the existing technology, the invention provides a production method of entacapone tablets, including the following steps:

Step S1: the auxiliary material is screened: the auxiliary material microcrystalline cellulose, mannitol and cross-linked carboxymethylcellulose are screened for 80 mesh for standby;

Step S2, ingredients: weigh the pre ordered microcrystalline cellulose, mannitol and cross-linked carboxymethylcellulose respectively according to the proportion of 10:9:4 of the auxiliary materials in step S1 for standby:

Step S3, mixing: put the auxiliary material from step S2 and entacapone in a number of motion mixers, mix at 25Hz speed for 5 minutes;

Step S4, screening: the material obtained in step S3 is screened for 20 mesh for standby;

Step S5, mixing: place the material obtained in step S4 in a number of motion mixers and mix at 25Hz for 10 minutes:

Step S6, dry method particle: the mixed materials in step S5 are dried and pressed into the dry granulator equipped with 12 mesh whole screen, and the particles are screened by 20 and 60 sieves;

Step S7, screening: the prepared particles are passed through the shaker screen with 20 mesh and 60 mesh stainless steel screen, and the coarse particles of more than 20 mesh are added into the whole grain chamber of dry granulating machine from the whole grain groove, and the whole grain is continued. The fine powder under 60 mesh is re pelletized, so that the fine powder amount accounts for about 15% of the total material of the dry method grain, and then stop the granulation;

Step S8: put the material obtained in step S7 and the predetermined amount of magnesium stearate into a number of motion mixers, and mix at 25Hz for 10 minutes;

Step S9: the finished product is made by pressing the material obtained in step S8.

Wherein, in step S1, mannitol is prepared by the following steps:

Step S11: the mixture of fructose and glucose was hydrolyzed from sucrose;

Step S12: further hydrolyze the obtained glucose to obtain a mixture of mannose, fructose and glucose;

Step S13: the mannose, fructose and glucose obtained in step S11 and step S12 are mixed together, hydrorefining, concentration, crystallization and pressure filtration are added to obtain the mannitol finished product;

Step S14: the finished mannitol product is dried and crushed by mixing with water mixed with binder, and the compressed mannitol particles with a particle size of 200-350 um are obtained, which are used as auxiliary materials for the preparation of entacapone tablets.

Wherein, step S11 includes the following steps:

S11a: take sucrose, prepare sugar solution with a mass concentration of 40%-60%, adjust the pH between 3-4.5, add molybdate catalyst, and react at 95-100° C. for 1 hour, so that the sucrose solution can be hydrolyzed completely;

Step S11b: the hydrolyzed solution is exchanged by ion exchange resin, and the converted sugar including a mixture of fructose and glucose is obtained;

Step S11c: the transformed sugar was separated from the simulated mobile bed to separate fructose and glucose. The separation temperature was between 55-65° C., and the adsorbent was selected from calcium cation exchange resin and eluent was selected from water.

Wherein, step S12 includes the following steps:

Step S12a: molybdate catalyst was added to the separated glucose solution, the pH was adjusted to be between 4.5-5.5, and the differential isomerization was 4-5 hours at 90-105° C.;

Step S12b: the solution after differential isomerization was exchanged by ion exchange resin, and then separated by simulated mobile bed. The mannose and fructose obtained after isomerization of glucose and glucose were separated. The separation temperature was between 55-65° C., and the adsorbent was selected from calcium cation exchange resin and eluent was selected from water;

Step S12c: repeat the differential isomerization in step s12a again for the separated glucose solution.

Wherein, step S13 includes the following steps:

Step S13a: after hydrogenation reaction, ion exchange resin exchange and reverse osmosis membrane preconcentration, concentration, pre crystallization and filtration, solid mannitol crude products and mother liquor are obtained by hydrogenation reaction, ion exchange resin exchange and reverse osmosis membrane preconcentration, concentration, pre crystallization and pressure filtration;

Step S13b: after separating mother liquor in simulated mobile bed, sorbitol and mannitol are obtained. After the separated sorbitol is preconcentrated, concentrated, pre crystallized and pressed filtered by reverse osmosis membrane, solid mannitol crude product is obtained:

Step S13c: the crude mannitol product was further refined to produce the mannitol finished product.

Wherein, step S14 includes the following steps:

Step S14a: add the finished mannitol product into the water with the binder dissolved, stir it fully, and the water content is between 30-40% of the finished mannitol product dosage;

Step S14b: the mixed material was put into a vacuum dryer to dry, the drying temperature was between 70-75° C. and the drying time was between 2-3 hours;

Step S14c: crush and screen the vacuum dried material to obtain the compressible mannitol particles.

Wherein, in step S3, entacapone is prepared by the following steps:

Step S31, nitrification, includes:

Step s31a: add glacial acetic acid and vanillin into the reaction tank, drop 65% nitric acid at 15-30° C., react for 3-5 hours at 20-30° C., add purified water for 1 hour, and the ratio of reactant mass is as follows: 1 part of vanillin, 0.8 parts of 65% nitric acid, 1.8-2.2 parts of glacial acetic acid;

Step S31b: centrifugal filtration;

Step S31c: the intermediate nitrovanillin was obtained after air drying for 12-14 hours at 40-50° C.;

Step S32, demethylation, includes:

Step S32a: add nitrovanillin, dichloromethane, tetrabutylammonium bromide to the reaction tank, add anhydrous aluminum chloride at 0-5° C., pyridine at 0-10° C., reflux reaction for 23-25 hours, add 3N hydrochloric acid, stir for 3 hours, and the ratio of reactant mass is as follows: 1 part of nitrovanillin, 3.3-3.5 parts of dichloromethane, 0.015-0.017 parts of tetrabutylammonium bromide Anhydrous aluminum chloride was 0.90-1.00 and pyridine 1.3-1.5;

Step S32b: centrifugal filtration;

Step S32c: 40-50° C. was dried for 10-13 hours, and the intermediate 3,4-dihydroxy-5-nitrobenzaldehyde was obtained;

Step S33, condensation reaction, including:

Step S33a: isopropanol, 3,4-dihydroxy-5 nitrobenzaldehyde, n-n-diethylcyanoacetamide and piperidine were added into the reaction tank. The reflux reaction was 22-24 hours. The ratio of reactants mass was as follows: 3.9-4. 1 parts of isopropanol, 3,4-dihydroxy-5 nitrobenzaldehyde 0.9-1.1 parts, n-n-diethylcyanoacetamide 0.90-0.95 parts, piperidine 0.55-0.57 parts:

Step S33b: after decompression and concentration, add glacial acetic acid and hydrochloric acid, stir at 20-25° C. for 19-21 hours, and add purified water at 10-15° C. for 2 hours:

Step S33c: centrifugal filtration;

Step S33d: the air blast drying was 14-16 hours at 35-45° C.:

Step S33e, acid solution: add dried material and glacial acetic acid into the reaction tank, raise temperature to 90° C., add hydrobromic acid with mass concentration of 40%, stir for 20 minutes and filter it into the crystallizer;

Step S33f: cool the material solution to 20-25° C. for 20 hours, then cool to 10-15° C. and stir for 5 hours;

Step S33g: centrifugal filtration;

Step S33h: dry the rough product of destamakubon at 40-50° C. for 4-6 hours;

Step S34, refining process, including:

Step S34a: add crude endacarbone and methanol into the crystallizer, return for 2 hours, cool to 5-10° C. and crystallize for 5 hours;

Step S34b: centrifugal filtration;

Step S34c: dry 9-11 hours at 40-50° C. and then dry the finished product of destamcapone.

The entacapone tablet prepared by the production method of the entacapone tablet provided by the invention has stable structure, stable physical properties and long shelf life.

### ILLUSTRATION OF THE INVENTION

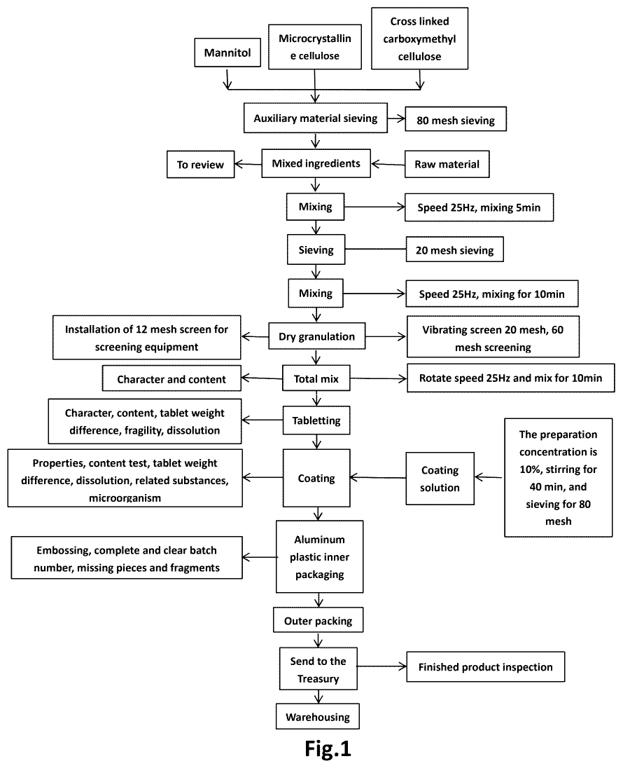

FIG. 1: process flow chart of the production method of entacapone tablets of the invention;

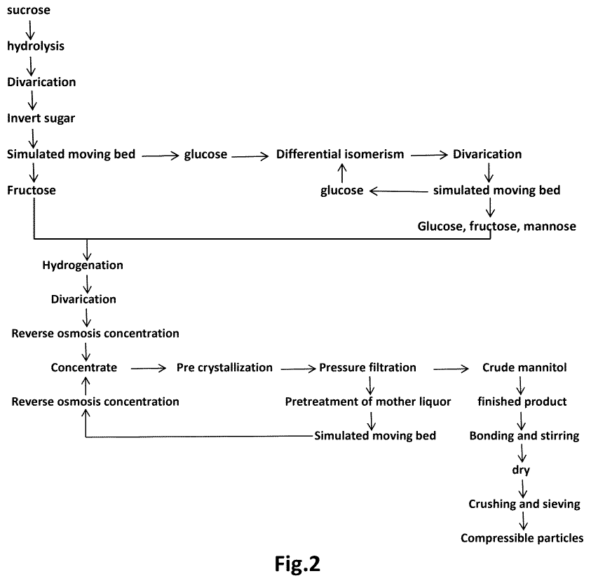

FIG. 2: flow chart of the production method of mannitol required by the invention.

### EMBODIMENT OF THE INVENTION

In order to have a further understanding of the technical scheme and beneficial effect of the invention, the technical scheme and the beneficial effect produced by the invention are described in detail below in combination with the attached drawings.

### I. Total Process

FIG. 1 is a process flow chart of the production method of the entacapone tablets provided by the invention, as shown in FIG. 1, the production method of the entacapone tablets provided by the invention includes the following steps:

Step S1. Screening of auxiliary materials: the auxiliary materials microcrystalline cellulose, mannitol and cross-linked carboxymethylcellulose are screened for 80 mesh for standby.

Step S2. Ingredients: the auxiliary materials in step SI are weighed as per the proportion of 10:9:4 to prepare the pre ordered microcrystalline cellulose, mannitol and cross-linked carboxymethylcellulose respectively.

Step S3. Mixing: place the auxiliary material from step S2 in a number of motion mixers with entacapone, mix at 25Hz for 5 minutes.

Step S4. Screening: the material obtained in step S3 is screened for 20 mesh for standby.

Step S5. Mixing: place the material obtained in step S4 in a number of motion mixers and mix at 25Hz for 10 minutes.

Step S6. Dry method particle: the mixed materials in step S5 are dried and pressed into the dry granulation machine equipped with 12 mesh whole screen, and the particles are screened by 20 and 60 sieves.

Step S7. Screening: Pass the particles prepared in step S6 through the shaker screen with 20 mesh and 60 mesh stainless steel screen, add the coarse particles of more than 20 mesh into the whole grain chamber of dry granulation machine from the whole grain groove, continue to complete the grain, and re granulation the fine powder under 60 mesh, so that the fine powder amount accounts for about 15% of the total material of the dry method grain, and then stop the granulation.

Step S8. The material obtained in step S7 and the predetermined amount of magnesium stearate are put into a number of motion mixers and mixed at 25Hz for 10 minutes.

Step S9. The finished product is made by pressing the material obtained in step S8.

In one of the better effect embodiments of the invention, the ratio of raw materials required in the process of tablet preparation is as follows:

Prescription

| | 1000 | 100000 |
|---|---|---|
| Material name | tablets | tablets |
| Entacapone | 200 g | 20000 g |
| Microcrystalline cellulose | 200 g | 20000 g |
| mannitol | 180 g | 18000 g |
| Crosslinked sodium | 80 g | 8000 g |
| carboxymethyl cellulose | | |
| Magnesium stearate | 10 g | 1000 g |

When preparing endacarbone tablets, the invention focuses on providing a preparation method of endacarbone tablets and mannitol. The quality of the products is guaranteed to the greatest extent by preparing raw materials by self-determination.

### II. Preparation of Mannitol

When preparing endacarbone tables, the compressibility between auxiliary material and main material will directly affect the quality of finished product. The purity and particle size of crystal mannitol directly affect its fluidity and compressibility. Therefore, when preparing the endacarbone sheet, the selected auxiliary mannitol is prepared independently through the following steps. As shown in FIG. 2, the preparation method of mannitol required in the invention:

### 1. Preparation of Complex Sugar From Sucrose

However, fructose is expensive and the yield of the product is low; A large number of by-products sorbitol will be mixed in the product by-product of the differential isomerization of glucose, and the yield of the product is lower; Mannitol can be directly prepared by mannose, but the source of mannose is limited.

The invention selects sucrose instead of traditional fructose or glucose to produce mannitol. The crystalline glucose contains crystalline water, and the weight of dry matter will be reduced after dissolving in water; The weight of dry matter increased after sucrose hydrolysis, and the price of sucrose raw material was lower than that of glucose, so it could save more cost to produce mannitol from sucrose.

The specific production steps are as follows:

(1) The sucrose was prepared into a sugar solution with a mass concentration of 40%-60%, the pH of which was adjusted to 3-4.5, molybdate catalyst was added to react for 1 hour at 95-100° C. to make the sucrose solution completely hydrolyzed;

(2) The hydrolyzed solution is exchanged by ion exchange resin, and the converted sugar of fructose and glucose mixture is obtained;

(3) The transformed sugar was separated from the simulated mobile bed to separate fructose and glucose. The separation temperature was between 55-65° C., and the adsorbent was selected from calcium cation exchange resin and eluent was selected from water.

The simulated mobile bed can be selected from any existing products. The 12 columns or 24 columns are connected at the head and tail. Each column is connected with the feed port, discharge port, water inlet and circulation inlet and outlet. The circulating pump is used to make the material flow in the column. The position of the feed port, discharge port, water inlet and circulating inlet and outlet can be changed by rotating valve or solenoid valve to achieve the purpose of separation. After the separation in simulated mobile bed, the fructose and glucose are about half of the mass. Fructose can be directly used for subsequent hydrogenation catalysis (detailed in the later article). Glucose needs to be further hydrolyzed through the next differential isomerization.

### 2. Glucose Heteroisomerism

The following steps are included:

(1) Molybdate catalyst was added to the separated glucose solution, and the pH was adjusted between 4.5-5.5 and the differential isomerization reaction was 4-5 hours at 90-105° C.;

(2) The solution after the differential isomerization was exchanged by ion exchange resin. The content of glucose component was about 10%, the content of mannose was about 90%, and a small amount of fructose was also contained; The mixed sugar solution was separated by simulated mobile bed, and the mannose and fructose obtained after isomerization of glucose and glucose were separated. The separation temperature was between 55-65° C., and the adsorbent was selected from calcium cation exchange resin and eluent was selected from water; The settings of the simulated mobile bed are the same as in step 1, and are not described here.

(3) The separated glucose solution was repeated the differential isomerization in step (2). The final compound sugar after one cycle could improve the content of mannose to 96% up and down, the glucose content was less than 4%, and there was a small amount of fructose.

That is, in this step, by simulating the setting of mobile bed, the conversion rate of glucose to mannose can be increased, the glucose content in the final compound sugar can be reduced, thus the content of sorbitol, the by-product in the final mannitol, and the yield and purity of the product can be improved.

### 3. Preparation of Mannitol Finished Products

The following steps are included:

(1) The solid mannitol crude product and mother liquor were obtained by hydrogenation reaction, ion exchange resin exchange and reverse osmosis membrane preconcentration, concentration, pre crystallization and filtration of fructose obtained in step 1 and the mixture of fructose, glucose and mannose obtained in step 2; Hydrogenation reaction and ion exchange resin exchange are mature technologies in the field of mannitol preparation, and the invention has no restrictions; It is important to note that the mixed liquid can generate mannitol liquid with high content after hydrogenation. The high content of mannitol is not conducive to subsequent ion exchange, concentration, crystallization and pressure filtration. Especially in low temperature environment, it is easy to crystallize, which causes equipment and pipeline blockage, The content of mannose needs to be controlled during hydrogenation: the content of mannose in the solution obtained by step 2 of the invention reaches almost 100%. Therefore, only the fructose content in step 1 and the mass ratio of the content of the mixed sugar solution obtained in step 2 need to be controlled, Compared with directly controlling the content of mannose in the mixed sugar solution obtained from the differential isomerization by controlling the process of differential isomerization, the invention creatively produces fructose and glucose by sucrose hydrolysis, then produces mannose by glucose hydrolysis, and then controls the amount of fructose and mannose as needed, which is obviously more convenient to operate.

The content of mannose in hydrogenation is controlled between 75-80%, which will not affect the subsequent preparation process, and reasonably control the production of by-products.

In addition, the invention can save the consumption of steam and reduce the production cost by pre concentration by reverse osmosis membrane and then steam concentration.

(2) After the mother liquor is separated by simulated moving bed, sorbitol and mannitol are obtained. After the separated mannitol is preconcentrated, concentrated, pre crystallized and filtered by reverse osmosis membrane, the solid mannitol crude product can be further processed as by-product (FIG. 2 is not seen); The setting and principle of the simulated mobile bed are the same as those above, the invention is no longer described, and the effect of the reverse osmosis membrane preconcentration is the same as that above, and the invention is no longer described;

(3) The yield of the final product can reach 85%.

### 4. Preparation of Compressible Mannitol Particles

The following steps are included:

(1) Add the finished mannitol product into the water with the binder dissolved, stir it fully, and the water content is between 30-40% of the finished mannitol product;

(2) The mixed material was put into a vacuum dryer to dry, the drying temperature was between 70-75° C. and the drying time was between 2-3 hours;

(3) The compressed mannitol particles were obtained by crushing and sieving the vacuum dried materials; After the above process setting and parameter control, the compressible mannitol particle size is between 200-350 um. The mannitol within the particle size range has excellent fluidity and compressibility, and can be used to prepare entacapone tablets with stable structure, stable physical properties and long shelf life.

### III. Preparation of Main Materials

The entacapone obtained by the invention is prepared by the following steps:

### 1. Nitration Reaction

(1) Add glacial acetic acid and vanillin into the reaction tank, drop 65% nitric acid at 15-30° C., react for 3-5 hours at 20-30° C., add purified water for 1 hour, and mix the reactant with the following mass ratio: 1 part of vanillin, 0.8 parts of 65% nitric acid, 1.8-2.2 parts of ice acetic acid;

(2) Centrifugal filtration;

(3) The intermediate nitrovanillin was obtained by air drying for 12-14 hours at 40-50° C.

### 2. Demethylation Reaction

(1) Add nitrovanillin, dichloromethane, tetrabutylammonium bromide to the reaction tank, add anhydrous aluminum chloride at 0-5° C., add pyridine at 0-10° C., reflux for 23-25 hours, add 3N hydrochloric acid, stir for 3 hours, and the mass ratio of reactants is as follows: 1 part of nitrovanillin, 3.3-3.5 parts of dichloromethane, 0.015-0.017 parts of tetrabutylammonium bromide Anhydrous aluminum chloride was 0.90-1.00 and pyridine 1.3-1.5;

(2) Centrifugal filtration;

(3) The intermediate 3,4-dihydroxy-5-nitrobenzaldehyde was obtained by air drying for 10-13 hours at 40-50° C.

### 3. Condensation Reaction

(1) Isopropanol, 3, 4-dihydroxy-5 nitrobenzaldehyde, n-n-diethylcyanoacetamide and piperidine were added into the reaction tank. The reflux reaction was 22-24 hours. The ratio of reactants was as follows: 3.9-4. 1 parts, 3, 4-dihydroxy-5 nitrobenzaldehyde 0.9-1.1 parts, n-n-diethylcyanoacetamide 0.90-0.95 parts, piperidine 0.55-0.57 parts:

(2) After decompression and concentration, add glacial acetic acid and hydrochloric acid, stir at 20-25° C. for 19-21 hours, and add purified water at 10-15° C. for 2 hours;

(3) Centrifugal filtration;

(4) The air blast drying at 35-45° C. is 14-16 hours;

(5) Acid solution: add dried material and glacial acetic acid into the reaction tank, and heat it to 90° C., add hydrobromic acid with a mass concentration of 40%, stir for 20 minutes and filter it into the crystallizer;

(6) The reaction time of the reaction is 20 hours when the liquid is cooled to 20-25° C., and then cooled to 10-15° C. for 5 hours;

(7) Centrifugal filtration;

(8) Dry for 4-6 hours at 40-50° C. and desapen;

### 4. Refining Process, Including

(1) Add the crude entacapone and methanol into the crystallizer, return for 2 hours, cool to 5-10° C. and crystallize for 5 hours;

(2) Centrifugal filtration;

(3) Blast dry for 9-11 hours at 40-50 degrees Celsius, and donthacapone finished.

The final product of entacapone is yellow green or yellow powder, the weight loss of drying is less than or equal to 0.5%, the residue of ignition is less than or equal to 0.1%, and the purity is more than or equal to 99.5%.

Although the invention has been explained by the better embodiments, it is not used to limit the scope of protection of the invention. Any person skilled in the art, without departing from the spirit and scope of the invention, still belongs to the scope of protection of the invention in relation to the above embodiments. Therefore, the scope of protection of the invention shall be subject to the scope defined in the claims.

1. A method for producing entacapone tablets, wherein said method comprises the following steps:
Step S1: the auxiliary material is screened: the auxiliary material microcrystalline cellulose, mannitol and cross-linked carboxymethylcellulose are screened for 80 mesh for standby;
Step S2, ingredients: weigh the pre ordered microcrystalline cellulose, mannitol and cross-linked carboxymethylcellulose respectively according to the proportion of 10:9:4 of the auxiliary materials in step S1 for standby;
Step S3, mixing: put the auxiliary material from step S2 and entacapone in a number of motion mixers, mix at 25 Hz speed for 5 minutes;
Step S4, screening: the material obtained in step S3 is screened for 20 mesh for standby;
Step S5, mixing: place the material obtained in step S4 in a number of motion mixers and mix at 25Hz for 10 minutes;
Step S6, dry method particle: the mixed materials in step SS are dried and pressed into the dry granulator equipped with 12 mesh whole screen, and the particles are screened by 20 and 60 sieves;
Step S7, screening: the prepared particles are passed through the shaker screen with 20 mesh and 60 mesh stainless steel screen, and the coarse particles of more than 20 mesh are added into the whole grain chamber of dry granulating machine from the whole grain groove. and the whole grain is continued. The fine powder under 60 mesh is re pelletized, so that the fine powder amount accounts for about 15% of the total material of the dry method grain, and then stop the granulation;
Step S8: put the material obtained in step S7 and the predetermined amount of magnesium stearate into a number of motion mixers, and mix at 25Hz for 10 minutes;
Step S9: the finished product is made by pressing the material obtained in step S8.

2. The method according to claim 1, wherein in the step SI, mannitol is prepared by the following steps:
Step S11: the mixture of fructose and glucose was hydrolyzed from sucrose;
Step S12: further hydrolyze the obtained glucose to obtain a mixture of mannose, fructose and glucose;
Step S13: the mannose, fructose and glucose obtained in step S11 and step S12 are mixed together, hydrorefining, concentration, crystallization and pressure filtration are added to obtain the mannitol finished product; and
Step S14: the finished mannitol product is dried and crushed by mixing with water mixed with binder, and the compressed mannitol particles with a particle size of 200-350 um are obtained, which are used as auxiliary materials for the preparation of entacapone tablets.

3. The method for producing entacapone tablets according to claim 2, wherein the step S11 comprises the following steps:
S11a: take sucrose, prepare sugar solution with a mass concentration of 40%-60%, adjust the pH between 3-4.5, add molybdate catalyst, and react at 95-100° C. for 1 hour, so that the sucrose solution can be hydrolyzed completely;
Step s11b: the hydrolyzed solution is exchanged by ion exchange resin, and the converted sugar including a mixture of fructose and glucose is obtained; and
Step s11c: the transformed sugar was separated from the simulated mobile bed to separate fructose and glucose. The separation temperature was between 55-65° C., and the adsorbent was selected from calcium cation exchange resin and eluent was selected from water.

4. The method for producing entacapone tablets according to claim 2, wherein the step S12 comprises the following steps:
Step s12a: molybdate catalyst was added to the separated glucose solution, the pH was adjusted to be between 4.5-5.5, and the differential isomerization was 4-5 hours at 90-105° C.;
Step s12b: the solution after differential isomerization was exchanged by ion exchange resin, and then separated by simulated mobile bed. The mannose and fructose obtained after isomerization of glucose and glucose were separated; the separation temperature was between 55-65° C., and the adsorbent was selected from calcium cation exchange resin and eluent was selected from water;
Step s12c: repeat the differential isomerization in step s12a again for the separated glucose solution.

5. The method for producing entacapone tablets according to claim 2, wherein the step S13 comprises the following steps:
Step S13a: after hydrogenation reaction, ion exchange resin exchange and reverse osmosis membrane preconcentration, concentration, pre crystallization and filtration, solid mannitol crude products and mother liquor are obtained by hydrogenation reaction, ion exchange resin exchange and reverse osmosis membrane preconcentration, concentration, pre crystallization and pressure filtration;
Step s13b: after separating mother liquor in simulated mobile bed, sorbitol and mannitol are obtained. After the separated sorbitol is preconcentrated, concentrated, pre crystallized and pressed filtered by reverse osmosis membrane, solid mannitol crude product is obtained; and
Step s13c: the crude mannitol product was further refined to produce the mannitol finished product.

6. The method for producing entacapone tablets according to claim 2, wherein the step S14 comprises the following steps:
Step S14a: add the finished mannitol product into the water with the binder dissolved, stir it fully, and the water content is between 30-40% of the finished mannitol product dosage;
Step s14b: the mixed material was put into a vacuum dryer to dry, the drying temperature was between 70-75° C. and the drying time was between 2-3 hours; and
Step s14c: crush and screen the vacuum dried material to obtain the compressible mannitol particles.

7. The method as claimed in claim 1, wherein, in step S3, entacapone is prepared by the following steps:
Step S31, nitrification, includes:
Step S31a: add glacial acetic acid and vanillin into the reaction tank, drop 65% nitric acid at 15-30° C., react for 3-5 hours at 20-30° C., add purified water for 1 hour, and the ratio of reactant mass is as follows: 1 part of vanillin, 0.8 parts of 65% nitric acid, 1.8-2.2 parts of glacial acetic acid;
Step S31b: centrifugal filtration;
Step S31c: the intermediate nitrovanillin was obtained after air drying for 12-14 hours at 40-50° C.;
Step S32, demethylation, includes:
Step S32a: add nitrovanillin, dichloromethane, tetrabutylammonium bromide to the reaction tank, add anhydrous aluminum chloride at 0-5° C., pyridine at 0-10° C., reflux reaction for 23-25 hours, add 3N hydrochloric acid, stir for 3 hours, and the ratio of reactant mass is as follows: 1 part of nitrovanillin, 3.3-3.5 parts of dichloromethane, 0.015-0.017 parts of tetrabutylammonium bromide Anhydrous aluminum chloride was 0.90-1.00 and pyridine 1.3-1.5:
Step S32b: centrifugal filtration;
Step S32c: 40-50° C. was dried for 10-13 hours, and the intermediate 3,4-dihydroxy-5-nitrobenzaldehyde was obtained;
Step S33, condensation reaction, including:
Step S33a: isopropanol, 3,4-dihydroxy-5 nitrobenzaldehyde, n-n-diethylcyanoacetamide and piperidine were added into the reaction tank. The reflux reaction was 22-24 hours. The ratio of reactants mass was as follows: 3.9-4.1 parts of isopropanol, 3,4-dihydroxy-5 nitrobenzaldehyde 0.9-1.1 parts, n-n-diethylcyanoacetamide 0.90-0.95 parts, piperidine 0.55-0.57 parts;
Step S33b: after decompression and concentration, add glacial acetic acid and hydrochloric acid, stir at 20-25° C. for 19-21 hours, and add purified water at 10-15° C. for 2 hours;
Step S33c: centrifugal filtration;
Step S33d: the air blast drying was 14-16 hours at 35-45° C.;
Step S33e, acid solution: add dried material and glacial acetic acid into the reaction tank, raise temperature to 90° C., add hydrobromic acid with mass concentration of 40%, stir for 20 minutes and filter it into the crystallizer;
Step S33f: cool the material solution to 20-25° C. for 20 hours, then cool to 10-15° C. and stir for 5 hours;
Step S33g: centrifugal filtration;
Step S33h: dry the rough product of destamakubon at 40-50° C. for 4-6 hours;
Step S34, refining process, including:
Step S34a: add crude endacarbone and methanol into the crystallizer, return for 2 hours, cool to 5-10° C. and crystallize for 5 hours;
Step S34b: centrifugal filtration;
Step S34c: dry 9-11 hours at 40-50° C. and then dry the finished product of destamcapone.
